(19) United States
(12) Patent Application Publication
(10) Pub. No.: US 2010/0160380 A1
(43) Pub. Date: Jun. 24, 2010

(54) Therapeutic Salt Compositions and Methods

(76) Inventors: Mark P. Rubino, Irvine, CA (US); Gyorgy F. Ambrus, Santa Ana, CA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/509,565

(22) Filed: Jul. 27, 2009

Related U.S. Application Data
(63) Continuation of application No. 11/744,036, filed on May 3, 2007, now abandoned.
Application No. 11/744,036 is a continuation-in-part of application No. 11/620,626, filed on Jan. 5, 2007, now abandoned.
(60) Provisional application No. 60/757,763, filed on Jan. 10, 2006.

(51) Int. Cl. A61K 31/4439; C07D 401/12

(52) U.S. Cl. 514/338; 546/273.7

(57) ABSTRACT

Therapeutic salt compositions and methods are disclosed herein.

### CROSS-REFERENCE TO RELATED APPLICATION

This application is a continuation-in-part of U.S. patent application Ser. No. 11/620,626, filed Jan. 5, 2007, which claims the benefit of U.S. provisional patent application Ser. No. 60/757,763, filed on Jan. 10, 2006, each of which is incorporated herein by reference in its entirety.

### DESCRIPTION

Sulfonyl ester prodrugs of proton pump inhibitors have been recently disclosed. For example, U.S. Pat. No. 6,897,227, expressly disclosed herein by reference, discloses such compounds. These compounds are designed to hydrolyze in vivo to yield the traditional proton pump inhibitors such as omeprazole, lansoprazole, pantoprazole, rabeprazole, or related compounds. However, the prodrugs are also susceptible to hydrolysis in vitro in aqueous solutions. The salt forms of the prodrugs have been prepared to facilitate formulation. Up to the conception of the presently disclosed invention, these compounds had been neutralized using weak bases and often organic cosolvents to avoid hydrolytic byproducts of the neutralization reaction. As a result, organic solvent impurities and weak acid impurities have been observed in the product salt.

Disclosed herein is a method of converting a carboxylic acid to a salt comprising,

adding an aqueous solution of a strong base to an aqueous mixture containing said carboxylic acid,

while maintaining the pH of the said aqueous mixture at no more than about 10,

wherein said carboxylic acid is a prodrug of a proton pump inhibitor having an arylsulfonyl leaving group, wherein said leaving group also has a substituent having a carboxylic acid functional group.

An “aqueous solution” is a solution having more than 50 mole percent water. An “aqueous mixture” is a mixture containing more than 50 mole % water.

In this method, the pH may also be maintained above about 3. Alternatively the pH may be above about 5. Alternatively, the pH may be above about 7. The pH is also maintained below about 10. Alternatively, the pH is maintained below about 9. Thus, although other pH ranges are possible, examples of pH ranges for the neutralization include from about 3 to about 10, from about 5 to about 9, and from about 7 to about 9.

An arylsulfonyl leaving group is —SO₂Ar, where the sulfur atom of the arylsulfonyl attaches to the nitrogen of the proton pump inhibitor. Ar is an aryl group, including a heteroaryl group, which includes, but is not limited to phenyl, naphthyl, thienyl, pyridinyl, and the like. Ar has at least one substituent, and at least one of the substituents has a carboxylic acid moiety.

In one embodiment, the carboxylic acid consists of

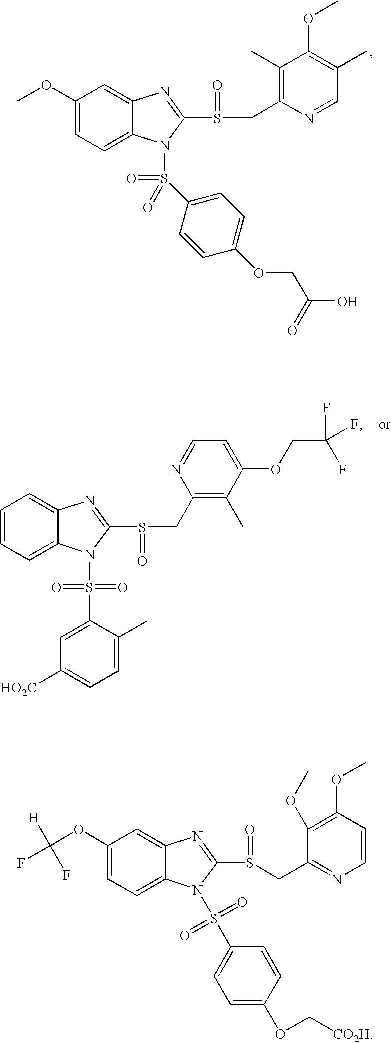

In one embodiment, the carboxylic acid is

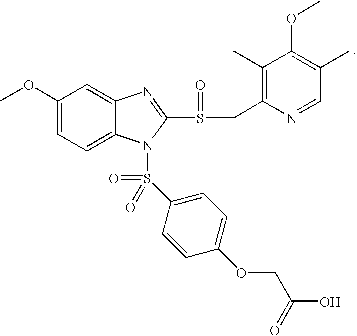

In another embodiment the salt is a sodium salt.

In another embodiment the salt is sodium {4-[5-Methoxy-2-(4-methoxy-3,5-dimethyl-pyridin-2-ylmethanesulfinyl)-benzoimidazole-1-sulfonyl]-phenoxy}-acetate.

A carboxylic acid is a compound having a CO₂H moiety. A carboxylic acid has two forms: 1) the acid or protonated form, and 2) the deprotonated, carboxylate ion, conjugate base, or anionic form.

A salt is an associated group of ions. In converting a carboxylic acid form to a salt, the carboxylic acid is deprotonated by a base such that the carboxylate ion is formed. One or more of the carboxylate ions are formally associated with one or more positively charged counterions, such as sodium, potassium, ammonium, or the like. But the salt may be dissolved and dissociated such that the counterion is not actually near the anionic CO₂—. Thus, the corresponding salt form of a carboxylic acid is the salt that is formed when the carboxylic acid is deprotonated by a base.

A strong base has the meaning generally understood in the art. In other words, a strong base is a base which reacts essentially completely with water to form OH—, or alternatively, dissociates essentially completely in water to yield free OH—. Examples include, but are not limited to:

Group 1A metal hydroxides such as sodium hydroxide, potassium hydroxide, lithium hydroxide, rubidium hydroxide, cesium hydroxide, and the like;

Group 2A metal hydroxides, such as calcium hydroxide, strontium hydroxide, barium hydroxide, and the like;

quaternary ammonium hydroxide;

Group IA and 2A amide salts, such as NaNH₂, KNH₂, KNHCH₃, and the like;

Imide salts; and

Group IA and 2A metal salts of alcohols.

In one embodiment, the temperature is maintained below about 30° C. while the base is added. In another embodiment, the temperature is maintained below about 22° C. while the base is added. The temperature must be high enough for the aqueous solution to be liquid. The melting point of an aqueous liquid is at or below 0° C., depending upon the concentration of dissolved material in the water. The freezing point depression can be determined by a person of ordinary skill in the art, or the freezing point of a liquid can be determined experimentally, but aqueous liquids are often at least −20° C. In another embodiment, the temperature is at least −10° C. In another embodiment the temperature is at least −5° C. In another embodiment, the temperature is at least 0° C.

The salts shown below are useful products of the processes disclosed herein, and are useful in the compositions and dosage forms disclosed herein. The names of the salts depicted are given below the corresponding structure.

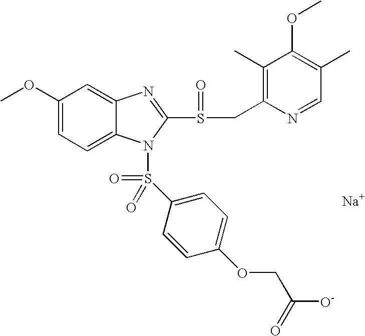

### Sodium {4-[5-Methoxy-2-(4-methoxy-3,5-dimethyl-pyridin-2-ylmethanesulfinyl)-benzoimidazole-1-sulfonyl]-phenoxy}-acetate

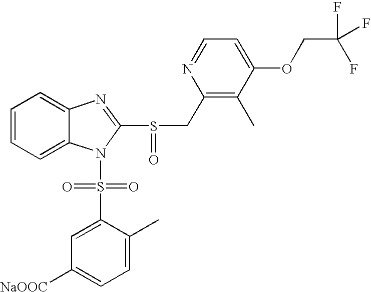

### Sodium 4-methyl-3-{2-[3-methyl-4-(2,2,2-trifluoro-ethoxy)-pyridin-2-ylmethanesulfinyl]-benzoimidazole-1-sulfonyl}-benzoate

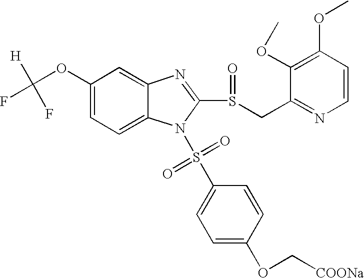

### Sodium {4-[5-Difluoromethoxy-2-(3,4-dimethoxy-pyridin-2-ylmethanesulfinyl)-benzoimidazole-1-sulfonyl]-phenoxy}-acetate

The present process facilitates neutralization of the carboxylic acid in greater quantities than was previously feasible. Thus, a composition consisting essentially of the carboxylic acid salt can be prepared, wherein the composition has a mass of from about 1 kg to about 10,000 kg. In other embodiments, the composition has a mass of from about 1 kg to about 1000 kg. In other embodiments, the composition has a mass of about 1 kg to 100 kg. In other embodiments, the composition has a mass of from about 7 kg to about 10,000 kg. In other embodiments, the composition has a mass of from about 7 kg to about 1000 kg. In other embodiments, the composition has a mass of about 7 kg to 100 kg. In other embodiments, the composition has a mass of from about 16 kg to about 10,000 kg. In other embodiments, the composition has a mass of from about 16 kg to about 1000 kg. In other embodiments, the composition has a mass of about 16 kg to 100 kg.

In one embodiment, greater than 1 kg of the carboxylic acid is used, neutralized, or converted in the described process. In another embodiment, greater than 7 kg of the carboxylic acid is used, neutralized, or converted in the described process. In another embodiment, greater than 16 kg of the carboxylic acid is used, neutralized, or converted in the described process.

In another embodiment, a further step in the process comprises spray drying an aqueous solution containing the salt form, the neutralized carboxylic acid, or the converted form of the carboxylic acid. In another embodiment, the aqueous solution that results from converting a carboxylic acid or neutralizing a carboxylic acid form is used directly in the spray drying process. In other words, no steps are taken on the solution between neutralizing or converting and spray drying.

In one embodiment, the carboxylic acid, which is obtained by the process described in U.S. Pat. No. 6,897,227, is dissolved or dispersed in water with vigorous stirring. A sodium hydroxide solution (0.34 M) is added slowly with continued stirring, such that the temperature is maintained between about 19° C. and 22° C., and the pH is maintained below about 10. When the pH exceeds about 10, addition of the sodium hydroxide is halted until the pH falls below about 10, when the addition is resumed. Addition is complete when the number of moles of sodium hydroxide added is equal to the number of moles of the carboxylic acid initially added to the mixture.

In another embodiment, the pH is maintained below about 9.

In one embodiment, no organic solvents are used during the process. Thus, compositions and dosage forms which are free of trace amounts of organic solvents are contemplated.

In another embodiment, no carbonate or bicarbonate is used in the process. Thus, compositions and dosage forms which are free of carbonate or bicarbonate are contemplated.

Another embodiment is a composition or dosage form containing less than 1% omeprazole on an active basis, i.e. less than 1% of the therapeutically active salt is omeprazole.

Unless otherwise indicated, % is intended to mean % w/w.

Another embodiment is a composition comprising a pharmaceutically acceptable salt of

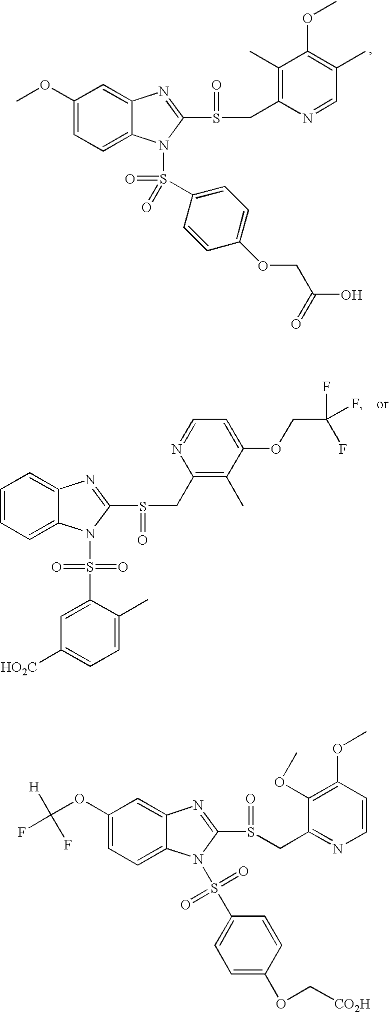

wherein said composition is at least about 96% pure on an anhydrous basis.

Another embodiment is a composition consisting of an essentially pure pharmaceutically acceptable salt of

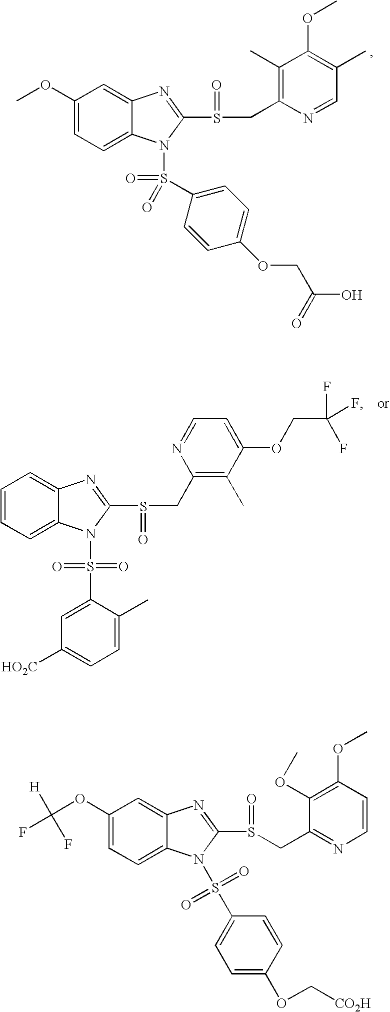

wherein said composition contains no ethyl hexanoic acid or acetonitrile.

Another composition consists essentially of pure

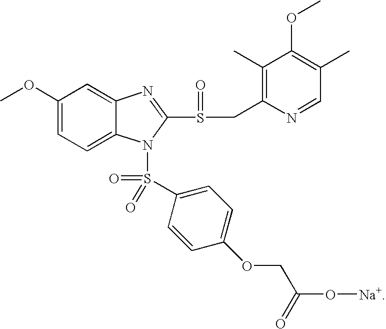

Another composition consists essentially of pure

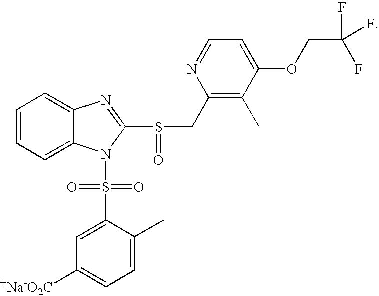

Another composition consists essentially of pure

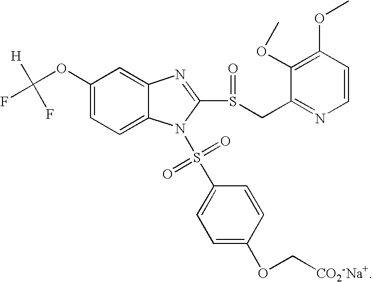

Another embodiment is a dosage form prepared by a process comprising

reacting a carboxylic acid form of a therapeutically active agent with an aqueous solution of a strong base to form the corresponding salt form, wherein the therapeutically active agent is maintained in an aqueous mixture having a pH which is no more than about 10; and

combining said salt form with a pharmaceutically acceptable excipient;

said carboxylic acid form has a formula chosen from

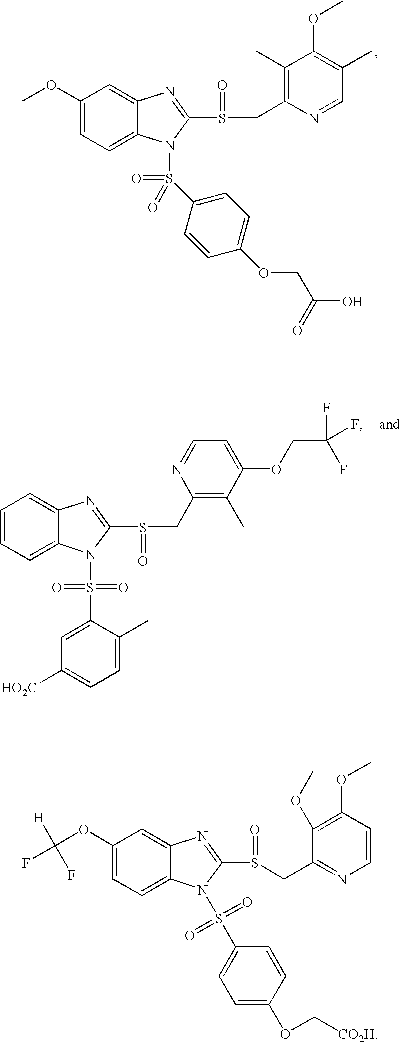

In another embodiment, the dosage form is prepared in a process which further comprises spray drying the aqueous mixture of the salt form.

Another embodiment is a dosage form comprising a salt form of a therapeutically active agent having a structure chosen from

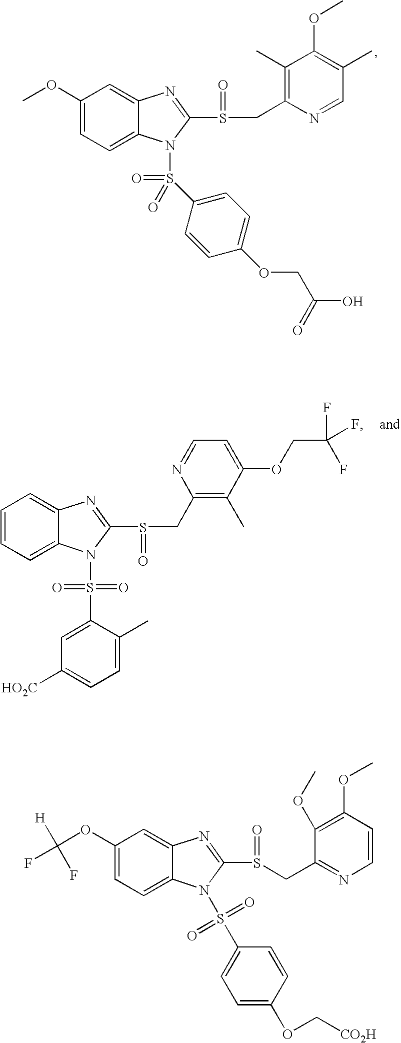

wherein said dosage form contains less than 107 parts per million of acetonitrile.

In another embodiment, the dosage form contains no acetonitrile.

Another embodiment is a dosage form comprising a salt form of a therapeutically active agent having a structure chosen from

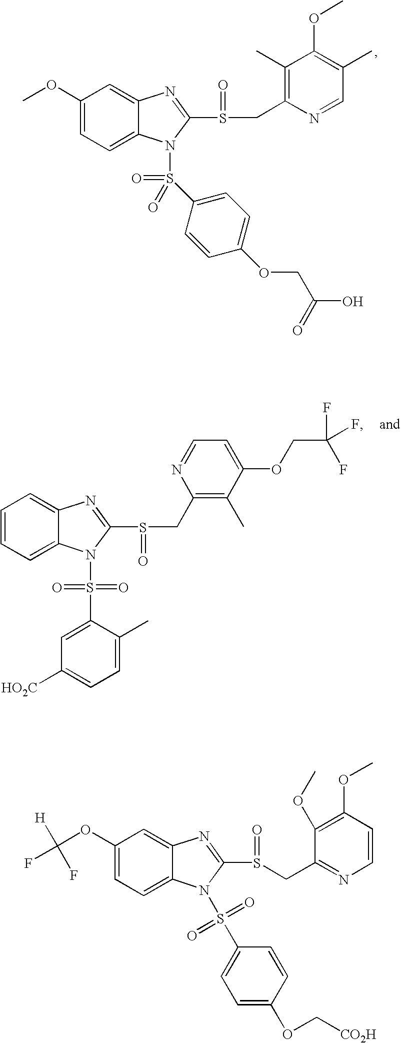

wherein said dosage form contains no ethyl hexanoic acid.

In another embodiment, the composition or dosage form contains no ethyl hexanoic acid.

In another embodiment, the composition or dosage form contains no acetonitrile.

In another embodiment, the composition or dosage form contains less than 107 parts per million of acetonitrile.

The methods disclosed herein may be useful in preparing dosage forms or compositions comprising a carboxylic acid salt which is free of one or more of the compounds shown below.

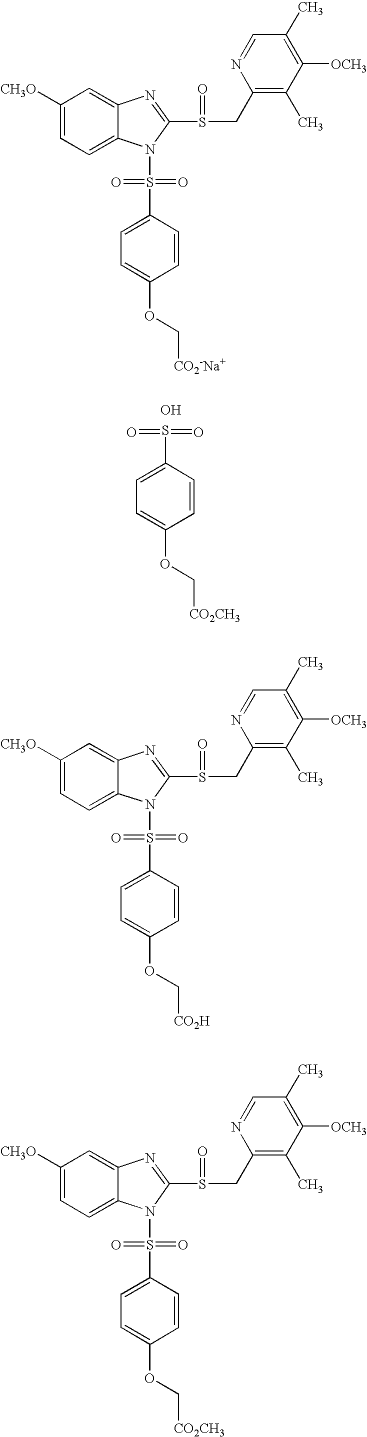

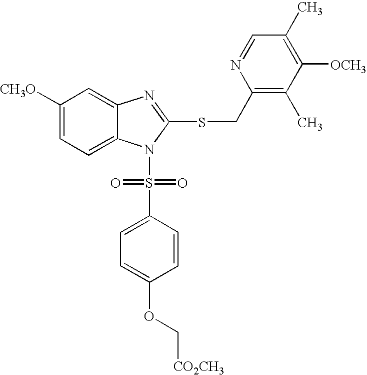

In Table 1 below, the impurity profile of a salt prepared by the process disclosed herein (G) is compared to the impurity profile of the same salt prepared using bicarbonate/carbonate or sodium ethyl hexanoate as the base and an organic solvent such as acetonitrile as a cosolvent (A-F). The structure of the salt is depicted below the Table.

TABLE 1

| Batch | A | B | C | D | E | F | G |
|---|---|---|---|---|---|---|---|
| Base Used | NaHCO₃ | NaHCO₃ | NaHCO₃ | NaHCO₃ | ethyl | ethyl | NaOH |
| | | | | | hexanoic | hexanoic | |
| | | | | | acid | acid | |
| HPLC purity (%) | 94.4 | 95.4 | 95.3 | 94.6 | 71.7 | 75.0 | 96.8 |
| Residual Sodium (ppm) | 44000 | 42500 | 47000 | 37000 | NA | NA | NA |
| Residual Acetonitrile (%) | 0.03 | 0.05 | 0.07 | 0.03 | NA | NA | 0 |
| ethyl hexanoic acid (%) | 0 | 0 | 0 | 0 | 3 | 8.5 | 0 |
| omeprazole | 0 | 0.2 | 0.2 | 0.6 | 6.0 | 6.6 | 0.7 |

NA Not available

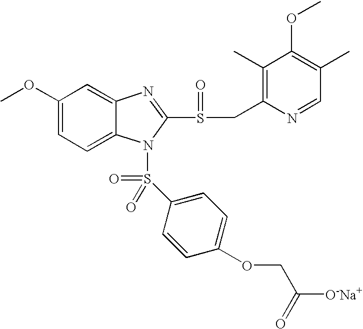

Another embodiment is a method of converting a carboxylic acid to a salt comprising,

adding an aqueous solution of a strong base to an aqueous mixture containing said carboxylic acid,
while maintaining the pH of the said aqueous mixture at no more than about 9, wherein said carboxylic acid consists of

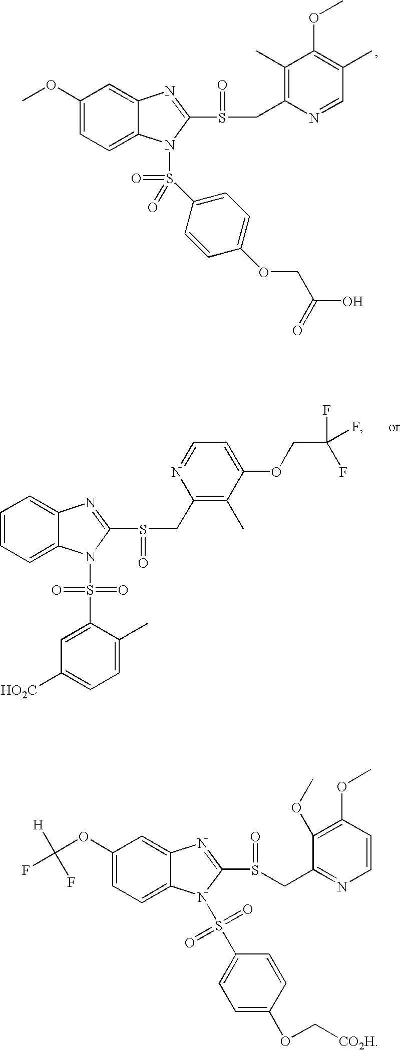

In another embodiment, the carboxylic acid is maintained at a temperature below about 30° C. while said base is added.

In another embodiment, wherein the carboxylic acid is maintained at a temperature below about 22° C. while said base is added.

In another embodiment, the carboxylic acid is

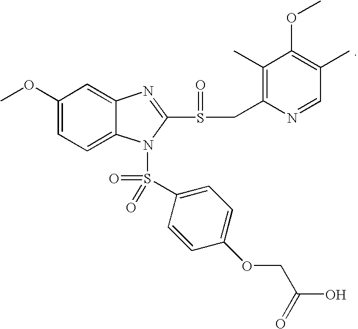

In another embodiment the salt is a sodium salt.

In another embodiment the salt is sodium {4-[5-Methoxy-2-(4-methoxy-3,5-dimethyl-pyridin-2-ylmethanesulfinyl)-benzoimidazole-1-sulfonyl]-phenoxy}-acetate.

In another embodiment greater than 1 kg of the carboxylic acid form is used.

Another embodiment is a composition consisting of an essentially pure pharmaceutically acceptable salt of

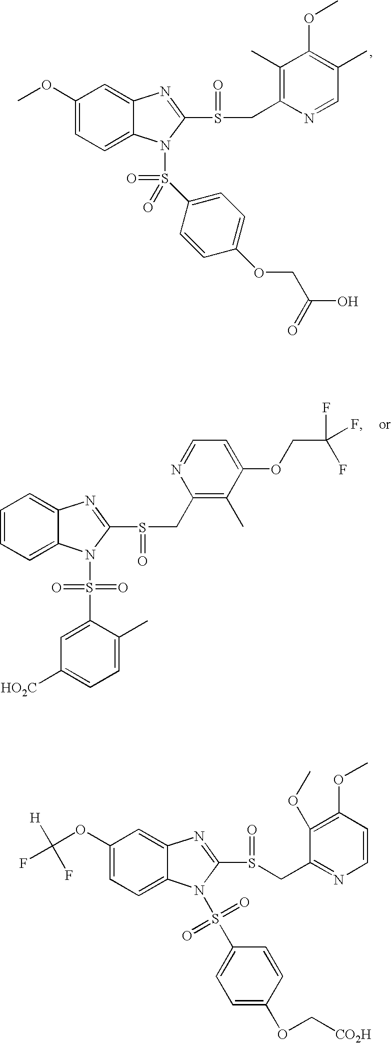

wherein said composition contains no ethyl hexanoic acid or acetonitrile.

Another embodiment is a composition consisting essentially of pure

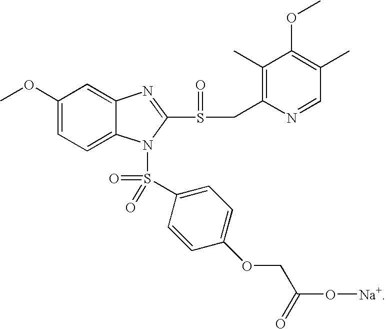

Another embodiment is a composition consisting essentially of pure

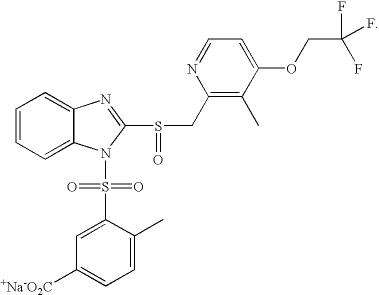

Another embodiment is a composition consisting essentially of pure

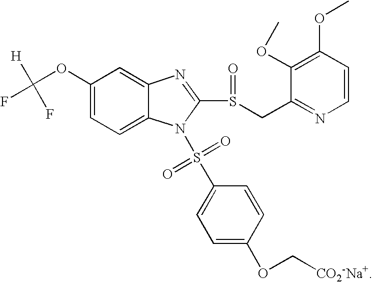

Another embodiment is a dosage form prepared by a process comprising

neutralizing a carboxylic acid form of a therapeutically active agent to its corresponding salt form using an aqueous solution of a strong base, wherein the therapeutically active agent is maintained in an aqueous mixture having a pH which is not more than about 9; and
combining said salt form with a pharmaceutically acceptable excipient;
wherein said carboxylic acid form has a formula chosen from

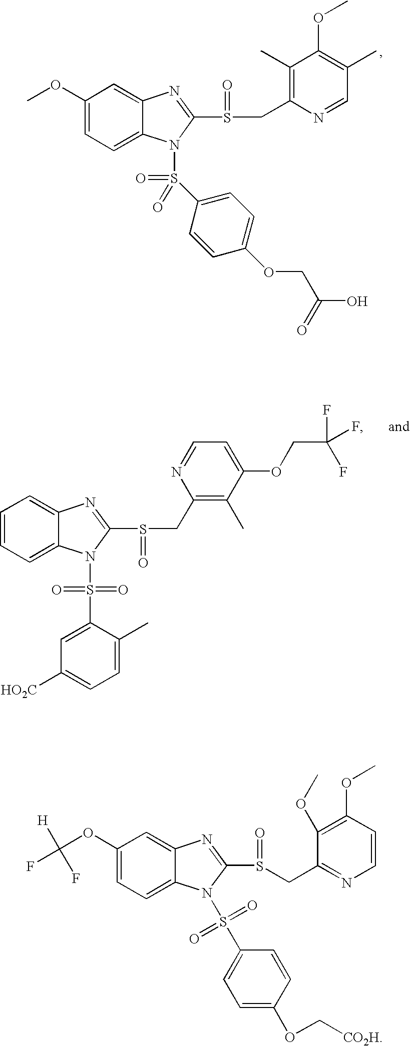

In another embodiment said process further comprises spray drying said aqueous mixture of said salt form.

Another embodiment is a dosage form comprising a salt form of a therapeutically active agent having a structure chosen from

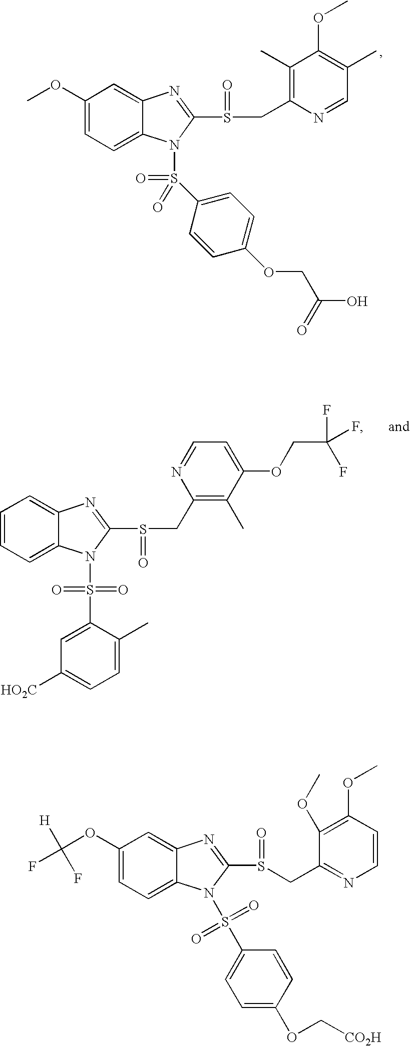

wherein said dosage form contains less than 107 parts per million of acetonitrile.

In another embodiment, the dosage form contains no acetonitrile.

Another embodiment is a dosage form comprising a salt form of a therapeutically active agent having a structure chosen from

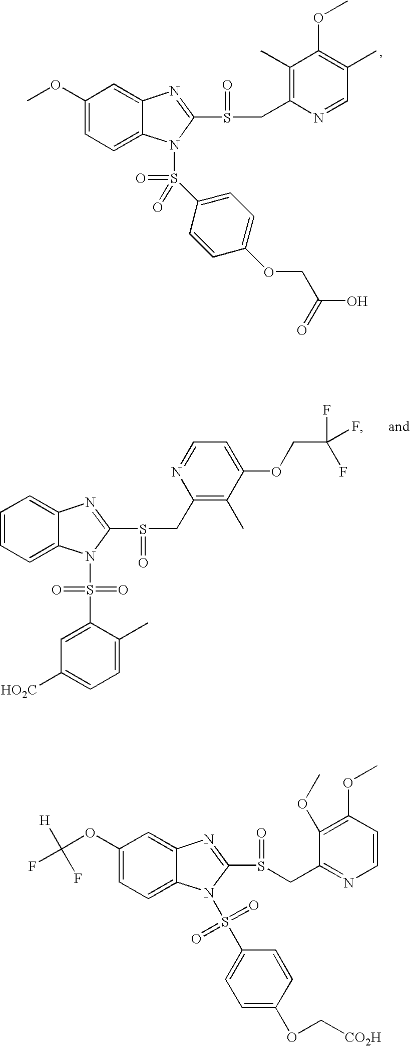

wherein said dosage form contains no ethyl hexanoic acid.

In another embodiment, the dosage form contains no acetonitrile.

Another embodiment is a dosage form comprising a salt form of a therapeutically active agent having a structure chosen from

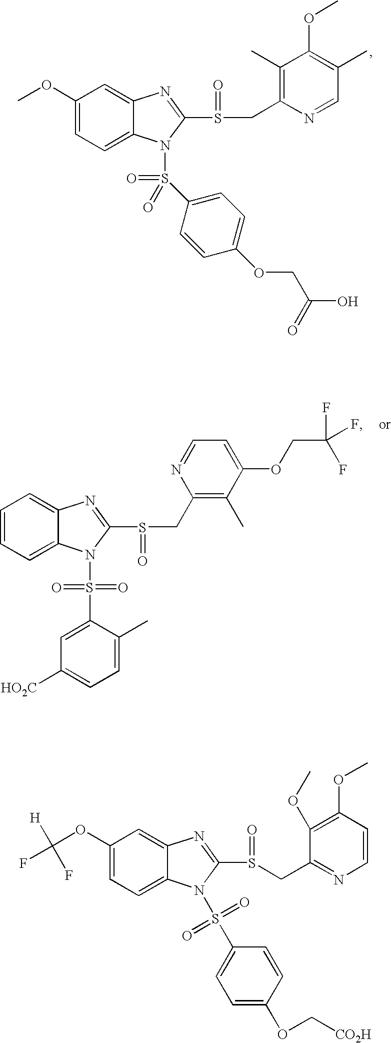

wherein the salt is greater than 96% pure on an anhydrous basis when it is used in the dosage form.

“On an anhydrous basis” means that the purity of a substance is what the purity of the substance is or would be when no water is present.

Another embodiment is a method of converting a carboxylic acid to a salt comprising,

adding an aqueous solution of a strong base to an aqueous mixture containing said carboxylic acid,
while maintaining the pH of the said aqueous mixture at no more than about 9, wherein said carboxylic acid is a prodrug of a proton pump inhibitor having an arylsulfonyl leaving group, wherein said leaving group also has a substituent having a carboxylic acid functional group.

Another embodiment is a composition, said composition having a mass of from about 1 kg to about 10,000 kg, wherein said composition consists essentially of

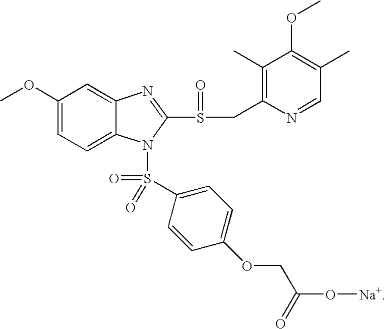

Although many specific embodiments are disclosed herein, they are merely examples, and none of these are intended to limit the scope of the invention in any way. The scope of the invention sought to be protected will be defined in the claims.

What is claimed is:

1. A method of converting a carboxylic acid to a salt comprising, adding an aqueous solution of a strong base to an aqueous mixture containing said carboxylic acid,
while maintaining the pH of the said aqueous mixture at no more than about 10,
wherein said carboxylic acid consists of

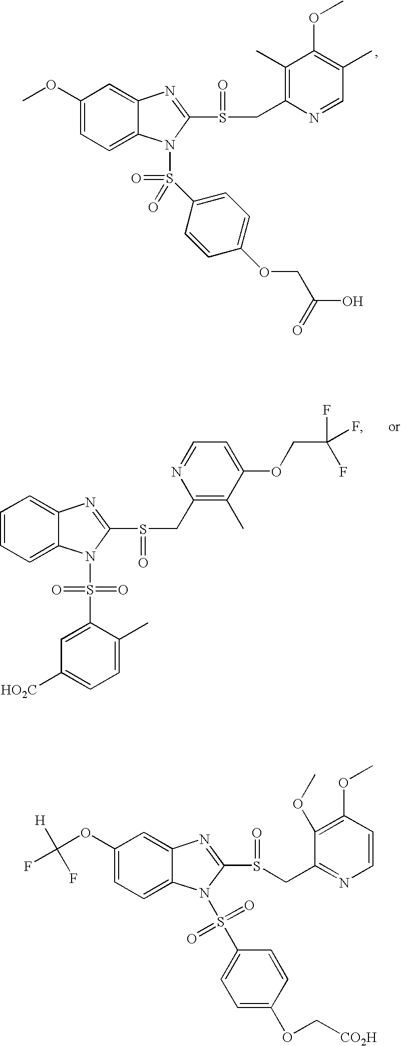

2. The method of claim 1 wherein the carboxylic acid is maintained at a temperature below about 30° C. while said base is added.

3. The method of claim 1 wherein the carboxylic acid is maintained at a temperature below about 22° C. while said base is added.

4. The method of claim 1 wherein the carboxylic acid is

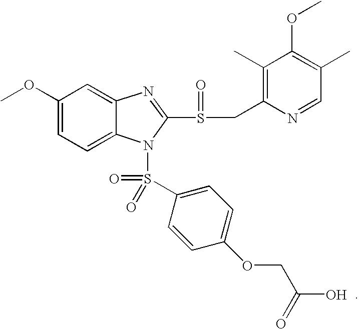

5. The method of claim 1, wherein the salt is a sodium salt.

6. The method of claim 5, wherein the salt is sodium {4-[5-Methoxy-2-(4-methoxy-3,5-dimethyl-pyridin-2-ylmethanesulfinyl)-benzoimidazole-1-sulfonyl]-phenoxy}-acetate.

7. The method of claim 1, wherein greater than 1 kg of the carboxylic acid form is used.

8. A composition consisting of an essentially pure pharmaceutically acceptable salt of 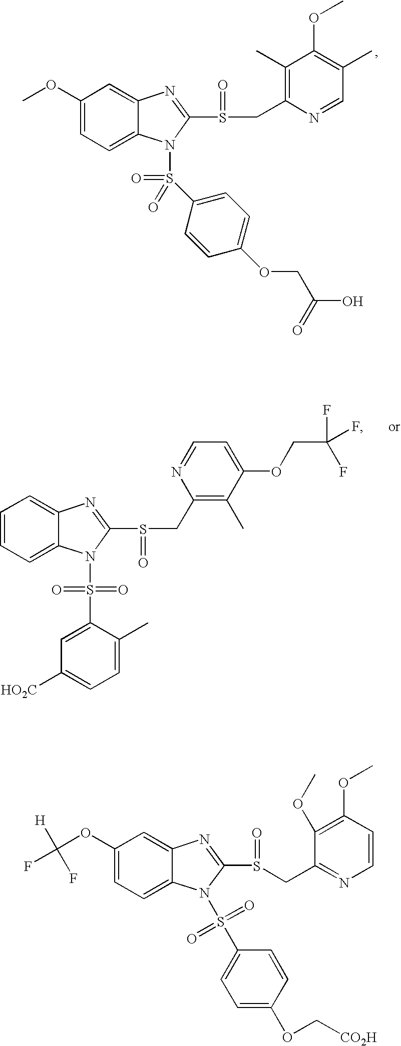
wherein said composition contains no ethyl hexanoic acid or acetonitrile.

9. The composition of claim 9 consisting essentially of pure

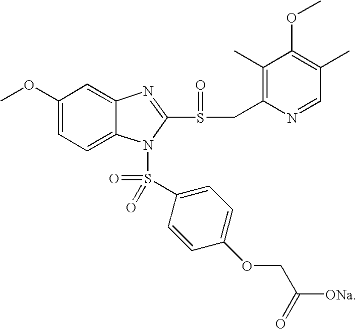

10. The composition of claim 9 consisting essentially of pure

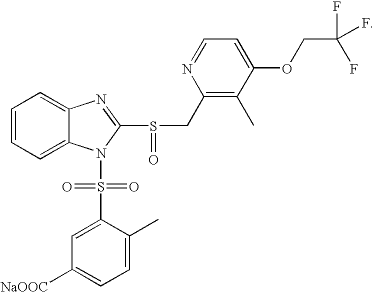

11. The composition of claim 9 consisting essentially of pure

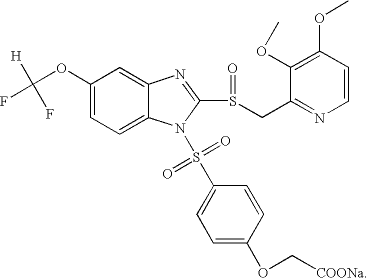

12. A dosage form prepared by a process comprising reacting a carboxylic acid form of a therapeutically active agent with an aqueous solution of a strong base to form the corresponding salt form, wherein the therapeutically active agent is maintained in an aqueous mixture having a pH which is no more than about 10; and
combining said salt form with a pharmaceutically acceptable excipient;
wherein said carboxylic acid form has a formula chosen from

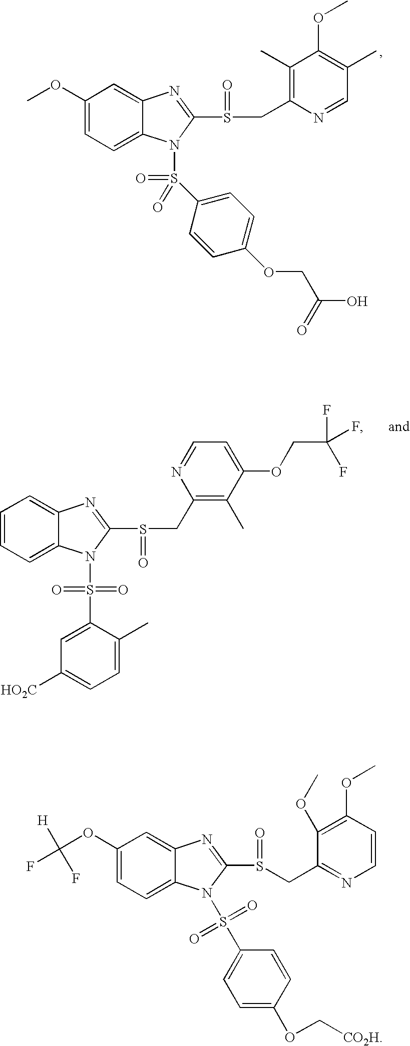

13. The dosage form of claim 9 wherein said process further comprises spray drying said aqueous mixture of said salt form.

14. A dosage form comprising a salt form of a therapeutically active agent having a structure chosen from 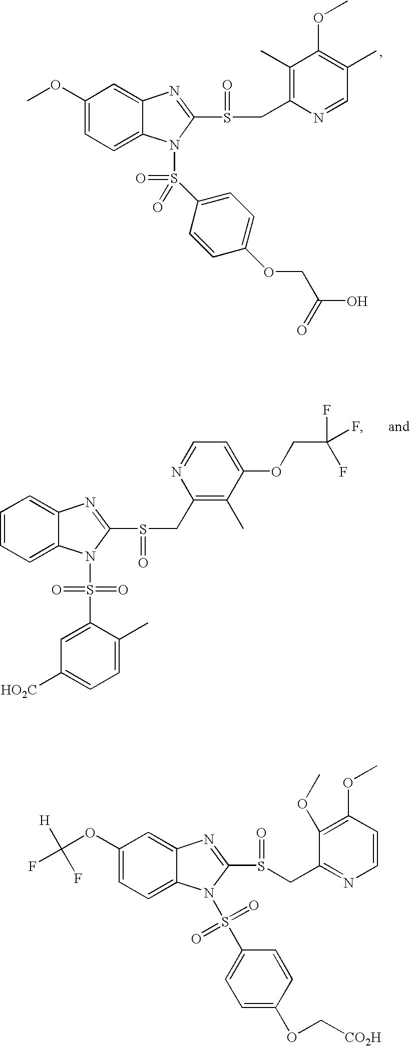
wherein said dosage form contains less than 107 parts per million of acetonitrile.

15. The dosage form of claim 14 which contains no acetonitrile.

16. A dosage form comprising a salt form of a therapeutically active agent having a structure chosen from 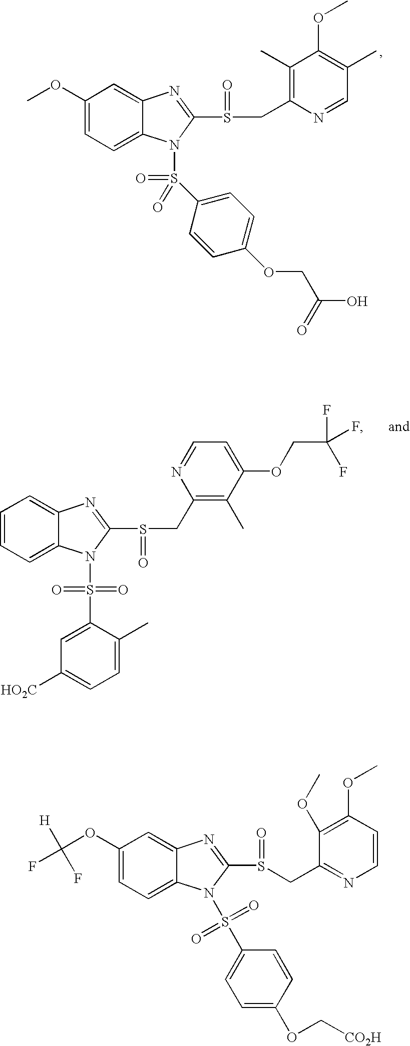
wherein said dosage form contains no ethyl hexanoic acid.

17. The dosage form of claim 17, which contains no acetonitrile.

18. A dosage form comprising a salt form of a therapeutically active agent having a structure chosen from 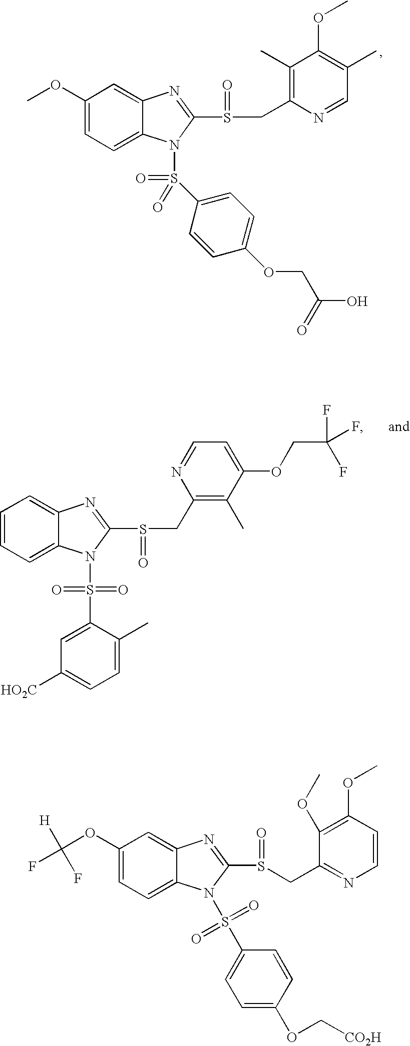
wherein the salt is greater than 96% pure on an anhydrous basis when it is used in the dosage form.

19. A method of converting a carboxylic acid to a salt comprising, adding an aqueous solution of a strong base to an aqueous mixture containing said carboxylic acid,
while maintaining the pH of the said aqueous mixture at no more than about 10,
wherein said carboxylic acid is a prodrug of a proton pump inhibitor having an arylsulfonyl leaving group, wherein said leaving group also has a substituent having a carboxylic acid functional group.

20. A composition, said composition having a mass of from about 1 kg to about 10,000 kg, wherein said composition consists essentially of

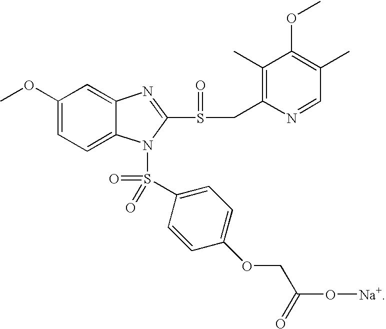

21. The method of claim 1 wherein the pH of the said aqueous mixture is maintained at no more than about 10.
